(19) United States
(12) Patent Application Publication
(10) Pub. No.: US 2018/0261333 A1
(43) Pub. Date: Sep. 13, 2018

(54) DRUG DISCOVERY PLATFORM

(71) Applicant: Anivive Lifesciences, Inc., Long Beach, CA (US)

(72) Inventors: Dylan Balsz, Los Angeles, CA (US); Cody Lloyd Arbuckle, Newport Beach, CA (US); David Bruyette, Woodland Hills, CA (US); Nicholas Sirago, Los Alamitos, CA (US)

(21) Appl. No.: 15/913,652

(22) Filed: Mar. 6, 2018

Related U.S. Application Data
(60) Provisional application No. 62/467,972, filed on Mar. 7, 2017.

(51) Int. Cl. G16H 70/40; G06F 17/30

(52) U.S. Cl. CPC G16H 70/40; G06F 17/30106; G06F 17/30979

(57) ABSTRACT

Described is a system for discovering potential veterinary medicines. The system can identify compounds used in human medicine that are candidates for repurposing. The system can use a software application to search for possible candidate compounds for treating animal disease. It can also search research data, for example, clinical trial data, to identify potential compounds for use in veterinary medicine. The system may rank sources and report the search results along with supporting evidence.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND OF THE INVENTION

### Field of the Invention

This disclosure relates to systems and methods for drug discovery, and, in particular, for discovering drugs for use in veterinary medicine.

### Description of the Related Art

A large percentage of veterinary diseases have no effective pharmaceutical treatments. As a result, there are millions of potentially preventable animal deaths each year. Few drugs are available to prevent these deaths because drug development has not kept pace with veterinary market growth and pharmaceutical demand. Consequently, there are many opportunities to repurpose drugs developed for human medicine for use in veterinary medicine. Repurposing pre-existing human drugs can reduce risk to animals, reduce cost, and reduce the time required to bring much needed veterinary drugs to market.

However, there are problems associated with the traditional selection and repurposing process. Finding drugs that are good candidates for repurposing can be challenging, expensive, and time intensive (often requiring hundreds of hours to identify a single viable candidate for repurposing). In some cases there are biological differences between humans and animals that make repurposing a drug ineffective even if the drug initially appears to be a good candidate for repurposing.

### SUMMARY OF THE INVENTION

This disclosure relates to systems and methods for drug discovery, and, in particular, to an integrated system to discover human medicines that may be suitable for use in a veterinary application.

One embodiment is a system for identifying potential veterinary medicines. The system can use a software application to search a variety of available data sources to identify possible veterinary medicine candidate compounds. The system can identify candidates used in human medicine that showed some promise as candidates for repurposing to veterinary medicine use. In one embodiment, the system may search veterinary trial data, or other research data, to identify potential candidates that showed efficacy in human or animal trials and that may be useful in veterinary medicine. In some embodiments, the system ranks identified candidates and provides supporting evidence alongside the search results. Additional embodiments of the disclosure are described below.

In a first aspect, an electronic system for discovering and evaluating potential veterinary medicines is provided, comprising a first database of indexed human medical information; a processor configured to execute instructions that perform a method comprising receiving search terms from a user comprising drug or medical indication data, generating a first search query from the search terms, querying the first database to identify candidate human drug information based on the first search query, analyzing the candidate human drug information to identify animal data relating to the human drug information, and displaying at least one source of the identified animal data to the user.

In an embodiment of the first aspect, querying the first database to identify candidate human drug information comprises querying a database of human gene information and animal gene information.

In an embodiment of the first aspect, reviewing the candidate human drug information to identify animal data relating to the human drug information comprises comparing the human gene information to the animal gene information.

In an embodiment of the first aspect, the processor is further configured to compare the gene sequence of interest to a reference human gene sequence.

In an embodiment of the first aspect, the processor is further configured to retrieve metadata for the at least one source, and wherein displaying the at least one source comprises displaying a source annotated with metadata.

In an embodiment of the first aspect, the metadata includes one or more information selected from a candidate name, a drug name, a molecular formula, a molecular structure diagram, a mechanism of action, a biomolecule implicated in the medical indication, a therapeutic target, a medical indication for the animal, a medical indication for a human, a form factor, a mode of administration, pharmacokinetics, toxicology, adverse effects, patent information, intellectual property ownership data, researchers, authors, contact information of owners or licensees, a clinical testing report, a phase of regulatory approval, a type or class of drug, genetic data associated with the drug, a summary of drug related data, a sentiment report, efficacy data, supporting publications, business funding, business expenditures, design of experiment, results of clinical testing, regulatory submissions, regulatory documentation, and drug vendors.

In an embodiment of the first aspect, the processor is further configured to receive a drug candidate selection and display metadata associated with the drug candidate.

In an embodiment of the first aspect, the animal data is dog data or cat data.

In an embodiment of the first aspect, the processor is further configured to generate a first page ranking of sources and display the first page ranking.

In an embodiment of the first aspect, the processor is further configured to prepare a meta analysis from metadata for the first source and metadata for the second source, and display a result of the meta analysis.

In an embodiment of the first aspect, the at least one source is selected from the group consisting of a patent source, a news source, a business information source, a clinical trial source, a regulatory source, a dictionary source, and a research publication source.

In an embodiment of the first aspect, the system further comprises an index storing key words for sources in the first database, and wherein querying the first database comprises locating the at least one key word in the index.

In a second aspect, a method for discovering and evaluating potential veterinary medicines is provided, comprising receiving search terms from a user comprising drug or medical indication data, generating a first search query from the search terms, querying a first database to identify candidate human drug information based on the first search query, analyzing the candidate human drug information to identify animal data relating to the human drug information, and displaying at least one source of the identified animal data to the user.

In an embodiment of the second aspect, querying the first database to identify candidate human drug information comprises querying a database of human gene information and animal gene information.

In an embodiment of the second aspect, reviewing the candidate human drug information to identify animal data relating to the human drug information comprises comparing the human gene information to the animal gene information to determine gene homology between the animal gene data and the human gene data.

In an embodiment of the second aspect, querying a first database comprises querying an index associated with the first database.

In an embodiment of the second aspect, analyzing the candidate human drug information comprises ranking pages of data from the retrieved animal data relating to the human drug information.

In an embodiment of the second aspect, analyzing the candidate human drug information comprises retrieving metadata relating to the candidate human drug data and then displaying that metadata to the user.

In an embodiment of the second aspect, the metadata is selected from a drug candidate name, a drug name, a molecular formula, a molecular structure diagram, a mechanism of action, a biomolecule implicated in the medical indication, a therapeutic target, a medical indication for the animal, a medical indication for a human, a form factor, a mode of administration, pharmacokinetics, toxicology, adverse effects, patent information, intellectual property ownership data, researchers, authors, contact information of owners or licensees, a clinical testing report, a phase of regulatory approval, a type or class of drug, genetic data associated with the drug, a summary of drug related data, a sentiment report, efficacy data, supporting publications, business funding, business expenditures, design of experiment, results of clinical testing, regulatory submissions, regulatory documentation, and drug vendors.

In an embodiment of the second aspect, displaying at the least one source of the identified animal data comprises displaying an ordered list of the identified animal data.

For purposes of summarizing the invention and the advantages achieved over the prior art, certain objects and advantages are described herein. Of course, it is to be understood that not necessarily all such objects or advantages need to be achieved in accordance with any particular embodiment. Thus, for example, those skilled in the art will recognize that the invention may be embodied or carried out in a manner that can achieve or optimize one advantage or a group of advantages without necessarily achieving other objects or advantages.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 is a block diagram of one embodiment of a drug discovery system that is linked to a plurality of data and information sources for identifying potential veterinary medicine products.

FIG. 2 is a block diagram of the drug discovery system from FIG. 1 and includes example components and modules included therein.

FIG. 3 is flowchart of one embodiment of a method for discovering veterinary medicine candidates.

FIG. 4 is flowchart of one embodiment of a method for analyzing a gene sequence as part of identifying veterinary medicine candidates.

FIG. 5 depicts a display of an annotated source including metadata.

FIG. 6 is a screen capture of search results generated by embodiments of the drug development system.

### DETAILED DESCRIPTION

One embodiment is a Drug Discovery (DD) system for identifying potential veterinary medicines. The DD system can use one or more software applications to search stored databases of information for possible candidate compounds for treating animal disease. The DD system is designed to identify human drugs for use in veterinary medicine. The system can leverage existing information on human drugs to gather and analyze that that may indicate veterinary medicine candidates. For example, the system may input and analyze patents and patent terms, regulatory data, therapeutic target data, genetic data, clinical efficacy data from published clinical trials, safety/toxicology data, chemistry data, manufacturing and control (CMC) data, pharmacokinetic information, and public mentions of the candidate compound in the press, as well as the entities (who may be individuals or organizations) associated with the candidate compounds. These entities may include clinical and pharmaceutical researchers, corporate owners, assignees, licensees, and their interconnection via social networks. This full review of data associated with the candidate compound may significantly reduce the amount time required to discover candidate veterinary medicines.

The candidate compound may be a small molecule drug or a biological product. The candidate compound may also be, for example, a compound or a formulation of one or more other compounds. Although the disclosure is primarily directed to a system for identifying medicines, the system may identify candidates in various categories of medicine products. The categories may include, for example, small molecule drugs, biologics, formulations of multiple drugs, particular methods of treatment, medical devices, or candidate products that have aspects of more than one of the aforementioned categories.

In one embodiment the DD system may be implemented as part of a mobile application as discussed more fully below. The system may identify key research entities, ownership entities, or potential licensing entities with respect to a particular candidate, or with respect to a group of candidates. The entities may be, for example, natural persons, business organizations, governmental organizations, or educational institutions.

In some embodiments, the DD system searches for online sources of the necessary data to perform its analysis. In one embodiment, the DD system uses at least one internet “spider” to crawl the internet, discovering and searching web pages, and collecting data from a variety of sources. The sources searched by the DD system spider may include various repositories available on a computer network such as the world wide web, or a local network such as an institutional network, as public or private repositories. As known, a spider, also sometimes called a “web crawler”, is a software program that fetches web pages, documents, and other files linked to the web pages. The DD system then collects and scans the content of the web pages, documents, and other files returned by the DD system spider to generate large-scale data stores and databases of the retrieved information. These data returned by the DD system spider can then be catalogued, indexed and stored locally by the DD system for later search and retrieval of information. The databases may be agnostic databases in the sense that the databases may be compiled without reference to any particular query.

In some embodiments, the spiders scan text-based data, audio data, graphical data, or video data from their target data source. Once the data is returned to the local DD system, natural language processing may be utilized for extracting and characterizing the underlying information and to create keyword indexes of the data from each data source. In some embodiments the spiders access public or open-source repositories of clinical trial data, basic science data, genetic data, or other research data. In some embodiments, the spiders access authorized private, closed-source, or subscription-based data repositories.

Once the data is stored in the DD system, the system may use artificial intelligence (AI) software or other programs and processes to analyze the data and identify relationships between a candidate compound and the people who may be able to facilitate successful licensing agreements. For example, the system may include data from social and professional networking sites to identify connections between patent owners, licensees and assignees. The system may also identify connections between entities associated with a source and an entity performing a search, such as the user.

The system may include an interface through which a user can enter search queries. For example, a user wishing to access the system may input terms to be searched. Terms to be inputted and searched can describe the disease or condition to be treated, symptoms to be treated, type of compound sought, form factor, mechanism of action, and mode or route of administration. The terms may include an animal of interest. The term related to the animal of interest may be a common name, a species, a genus, or a more general classifier. The animal of interest may be a dog (canis lupus). The animal of interest may be classified as a mammal. The animal of interest may further be selected from a cat, a chicken, a cow, a goat, a sheep, a rat, a llama, a pig, a guinea pig, a hamster, or a rabbit. The terms may also include an indication. The terms may also include a therapeutic target, a symptom, or a mechanism of action. The terms may also include a biomolecule such as a protein or enzyme implicated in the indication.

In some embodiments, the user may select a primary target species followed by a secondary target species, a tertiary target species, and so on. In various embodiments, during the input stage, a user will input the group or organization with which the user is associated.

The system may derive a search query from the terms to be searched. The search query may include key words derived from, or related to, the search terms. The database may include dictionary information in order to correlate search terms with key words. The system may then query the database. Querying the database may include searching the database sources index for the key words.

In various embodiments, the system, in response to a user initiated search, will extract and organize data from the sources, and display the extracted data in a first reporting step. The first reporting step may include displaying an annotated source include metadata. In the first reporting step, search result data may be processed to have a visual component to aid the user in implementing criteria and/or filters to further refine the data. The search result data may be shown to the user in a graphical user interface (GUI) or dashboard that is interactive with the user. The GUI or dashboard may display the major attributes of the sources and/or drugs that were found in the search, as based on the information such as terms provided by the user during the inputting step.

The system may provide search result data including a set of ranked sources. The sources may be, for example, electronic publications such as document files, or web pages. The sources may include patent information, regulatory status information, clinical trial information, science information such as, for example, indication or therapeutic effect information, financial information, or other information described herein. The sources may be ranked based on a number of criteria relating to for example, therapeutic efficacy, regulatory approval status, or patent term.

In some embodiments, a user may select a source returned in the search. The system may then retrieve and/or generate metadata for the source. The metadata to be displayed can include a candidate name, such as a drug name, a molecular compound or formula, a molecular structure diagram, a mechanism of action, a biomolecule such as a protein or enzyme implicated in the indication, a therapeutic target, an indication for animals and/or humans, a form factor, a mode of administration, pharmacokinetics, toxicology, adverse effects, patent information, intellectual property ownership data, researchers, authors, contact information of owners or licensees, phase of clinical testing or regulatory approval, type or class of drug, genetic data associated with the drug, a summary of drug related data, general concerns, efficacy, supporting publications, business funding, business expenditures, design of experiment, results of clinical testing, regulatory submissions, regulatory documentation, and drug vendors.

In various embodiments, the system will generate and display an individual candidate overview. The candidate overview may be displayed in response to the inputting the name of the drug, e.g., by selecting a graphical or text element. The data to be displayed can include the drug name, the molecular compound, a molecular structure diagram, mechanism of action, indication for animals and humans, form factor, mode of administration, pharmacokinetics, toxicology, adverse effects, patent information, intellectual property ownership data, contact information of owners or licensees, phase of clinical testing or regulatory approval, type or class of drug, genetic data associated with the drug, a summary of drug related data, general concerns, efficacy, supporting publications, business funding, business expenditures, design of experiment, results of clinical testing, regulatory submissions, regulatory documentation, and drug vendors.

The database may be an agnostic database of sources including an index for sources therein. The system can identify medicines used in human medicine that are candidates for repurposing for use in veterinary medicine. The system can identify and page rank sources that disclose a candidate. The system can also search patent data and human or veterinary clinical trial data, and other research data, to identify clinical results for potential compounds for use in veterinary medicine. In some embodiments, the system ranks sources and generates metadata annotation of the sources. In some embodiments, the system includes a gene database for comparing a gene of an animal of interest to a corresponding human gene. Additional embodiments of the disclosure are described below.

As one example, the DD system may receive an input query from a user with the terms “canine” and “diabetes.” While real-time network searching may be undertaken, the system may generally operate by searching local stores of the data necessary to perform the search task. Thus, the DD system may first search an index created by natural language analysis of all U.S. and international patents. This database would include all text from all patents. Searching the patent information for “canine” and “diabetes” may return a series of patents that include these terms, ordered by their page rank according to how important these terms were to the patent's overall content. For example, the top ranked patents may include data from successful animal trials using a particular compound to treat diabetes in canines.

Once the DD system has identified the top ranked patents having these terms, it may then review the names of the inventors and assignees listed on the patents. From that data, the DD system may then execute additional searches to identify related data naming the same inventors. For example, research papers from the inventors discussing the animal work. Clinical trial data naming the inventors, public statements in the news media, or graduate thesis or other data may be scanned. In addition, the system may review the assignee data to determine the names of the technology transfer officers and licensing individuals if the assignee is a university. Because many universities publish their available technologies, the DD system may also review a University technology transfer website to determine if the technology may be available for license.

The system may perform additional extensive searches based on data that is discovered in the first level search. For example, any research papers returned from the inventors may be reviewed and the additional authors may be identified that were also working on canine diabetes. The DD system may then search for patents or clinical research data listing these other authors. If the additional authors are identified as being employed at a company or other university, those organizations may then be searched to determine if they have additional publications relating to canine diabetes.

The system may continue these additional extensible searches for a preset amount of time, or until a preset amount of data is returned to the DD system user so that the most amount of information is available relating to potential candidate compounds that could be used for veterinary medicine. Of course, it should be realized that the DD system does not need to start with only one data source such as the patent database mentioned above. The system has access to the downloaded data from multiple sources and may search indexes of their data simultaneously, or in serial, depending on the goals of the search and amount of data to be reviewed.

### System Overview

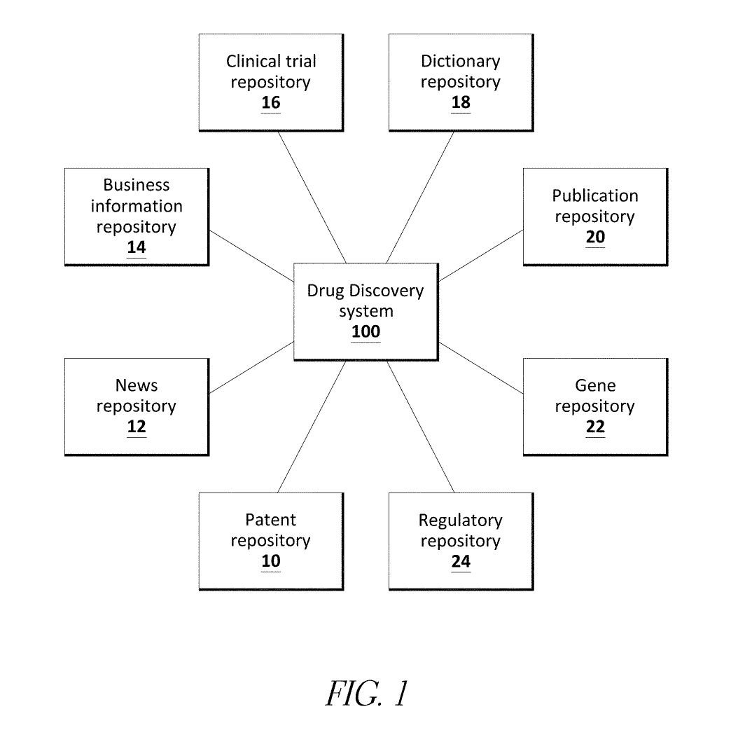

FIG. 1 is a block diagram that includes a drug discovery (“DD”) system 100 according to some embodiments. The system can acquire information from a number of repositories. In the illustrated embodiment of FIG. 1, these repositories include networked sources. For example, in FIG. 1, the DD system 100 communicates with data repositories that include a patent repository 10, a news repository 12, a business information repository 14, a clinical trial repository 16, a dictionary repository 18, a research publication repository 20, a gene data repository 22, and a regulatory information repository 24. Additional repositories are contemplated.

The patent repository 10 may include Espacenet, U.S.P.T.O. PAIR (United States Patent and Trademark Office Patent Application Information Retrieval), WIPO resources (World Intellectual Property Organization), China SIPO (State Intellectual Property Office), Google Patent Search, and other governmental and non-governmental patent resources. From this repository, the DD system may review and download some or all of the public information into the DD system databases for later searching.

The news repository 12 may include local data from newspapers, online newspapers, and news aggregators. The business information repository 14 may include the SEC (Securities and Exchange Commission) documents, state business databases, and other business information resources that can be searched and downloaded into the DD system. The clinical trial repository 16 may include FDA (Food and Drug Administration) resources and other governmental and non-governmental resources. The dictionary repository 18 may include general and specialist dictionaries, such as Webster's Dictionary, the Oxford Medical Dictionary, MedlinePlus, and the Merck Index. The research publication repository 20 may include PubMed, university libraries, and other governmental and non-governmental resources. The gene repository 22 may include GEO (Gene Expression Omnibus) databases, PUBMed database, and other governmental and non-governmental resources of gene information. The regulatory information repository 24 may include FDA (Food and Drug Administration) resources, EMA (European Medicines Agency) resources, and other governmental and non-governmental resources. Generally, the repositories will include information relating to human medicines and the use and study thereof. The repositories may also include information relating to animal medicines and the use and study thereof.

The repositories may provide information in any typical form. For example, the repositories may provide sources, which generally may include web pages, electronic documents, databases, spreadsheets, numerical information, graphical information, video information, or audio information. Each discrete page or document may be a source. In some embodiments, a source may correspond to a file or a linked group of files (for example, a linked set of web pages that make up a web site, or a text document with linked images).

A source may be a publication. The publications may include patents, scientific papers, theses, technical publications, submissions to an organization such as an oversight body, e.g., a government agency, government reports, marketing materials, generally promulgated information, and the like. The repositories may be available publically via the Internet. The repositories may also be available via private networks. The repositories may include governmental organizations, subscription services, or networks of institutions of learning. Generally, the system will access the repositories via automated processes, for example, web crawlers or spiders. The system may also be manually directed to access a repository. The system may access one or more repositories from time to time so that the system may be updated with new information.

### The Drug Discovery System

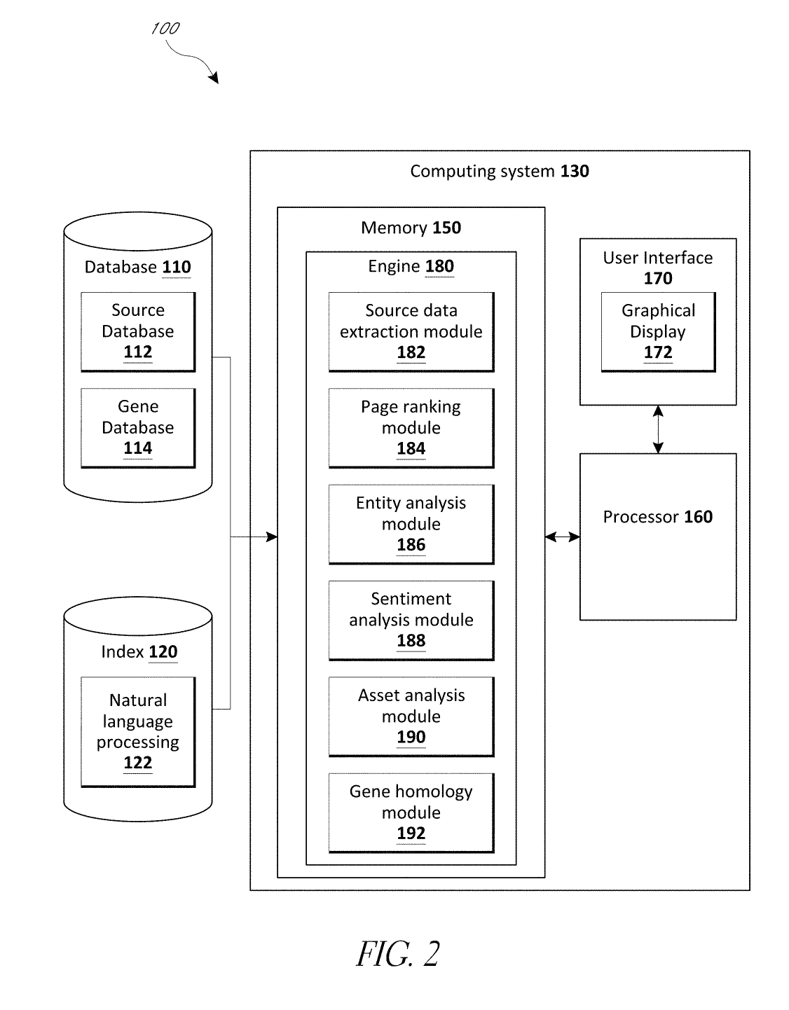

FIG. 2 is a schematic drawing that details additional components of the DD system 100. As shown, the DD system 100 includes a main database 110 that is configured to hold the data gathered from all the external sources and repositories shown in FIG. 1. The database includes a source database 112 and a gene database 114. Although these two databases are shown separately, it should be realized that the system may implement them in a single database, or separately, while still being encompassed within embodiments of the invention.

The database 110 can be a database of information, such as a database of raw data. The database 110 can comprise a single database or a plurality of databases. In an exemplary embodiment, the DD system 100 can include one or more databases including the source database 112 and the gene database 114. In some embodiments, the database 110 can store raw data. In some embodiments, the database 110 can store data that has been processed, such as by software to provide standard formatting. In some embodiments, the database 110 can store data that has been processed, such as by software to remove errors. The database 110 can be implemented to include additional data over time. The database 110 can include data going back up to 3 months, 6 months, 9 months, 1 year, 3 years, 5 years, 10 years, 20 years, 30 years, 60 years, 100 years, 500 years, or any range of any two of the foregoing values.

The database 110 can store additional information, such as information related to the functionality of the DD system 100. The database 110 can store one or more reports generated by the DD system 100. The database 110 can store any information relevant to the DD system 100 for any calculations, past, present, or future. The database 110 can store data generated during a user's previous interactions with the DD system 100. This can include the search entered by the user, the page ranked list of sources, asset reports, and any inputs made by the user. The DD system 100 can automatically, or as directed by the user, store data related to a user's interactions with the DD system 100. In some embodiments, the DD system 100 can customize future interactions between the DD system 100 and the user based on past interactions. For example, the DD system 100 can page rank sources according to the user's past interactions with the system.

The source database 112 stores sources that are indexed in index 120. A source may be a full-text source, by which is meant that the source is a rendering of all the information originally conveyed in the body of a source. Generally, the source database 112 stores at least some full text sources. In some embodiments, all or substantially all of the sources stored in the source database 112 may be full text sources. The source database 112 stores sources discovered while crawling the repositories. The sources may be compiled in the source database as processed by a computing system such as computing system 130. The computing system 130 may compress or archive the sources for storage in source database 112.

The index 120 may include data referencing the source database 112 and the gene database 114. The index 120 stores key words for sources stored in source database 112. Generally, index 120 will include a reference to a source stored in the source database 112. The index 120 may comprise the natural language processing module 122. The natural language processing module 122 may scan full text sources and analyze the text therein. The natural language processing module 122 may also perform a speech-to-text function, for example, for converting an audio clip into text for further processing and/or storage. The natural language processing module 122 may operate as known in the art.

Generally, when sources are compiled and stored in the source database 112, the source is scanned by the natural language processing module 122. The natural language processing module 122 may scan the source and extract key words from the source. The extracted key words may be stored in index 120. The natural language processing module may operate according to algorithms in the art. The natural language processing module may operate any function or functions to parse natural language text. For example, the natural language processing module may perform functions to determine the appropriate keywords within a retrieved data source. The natural language processor may be, for example, a suite comprising one or more of Stanford's Core NLP Suite, Natural Language Toolkit (NLTK), Apache Lucene, Apache Solr, Apache OpenNLP, GATE, or Apache UIMA.

The natural language processing module 122 may also perform a sentiment analysis on a full text source. The sentiment analysis may be used in part to develop a page ranking of sources in response to a user search, as described elsewhere herein.

The database 110 includes a gene database 114. The gene database stores genetic information. In particular, gene database 114 may store human genetic information and annotated genetic information for one or more animals. In some embodiments, gene database 114 stores the entire human genome. In further embodiments, gene database 114 stores an entire animal genome. In a particular embodiment, gene database 114 stores an entire human genome and an entire dog genome. Generally, the gene database stores information corresponding to sequences of base pairs found in DNA. The gene database may also store coding information and annotation for each gene in the database. Thus, the gene database may store information describing coding sequences for a protein. The gene database may also store mutation information, wherein the mutation information is correlated to indication or disorders arising from particular mutations.

The source database 112, gene database 114, and/or index 120 may be part of a physical storage medium such as a hard disk, optical disk, or solid state storage disk. The source database 112, gene database 114, and/or index 120 may be cloud based and may be physically remote from computing system 130.

In addition to being connected to the index 120, the database 110 is also linked to the computing system 130. In some embodiments, one or more of these components can be omitted. In some embodiments, the DD system 100 contains additional components not shown in FIG. 2. The DD system 100 can be embodied in a single device (e.g., a single computer or server) or distributed across a plurality of devices (e.g., a plurality of computers or servers).

The DD system 100 includes a general architecture of the computing system 130 configured to carry out the steps or methods for operating each of the modules discussed below. The general architecture of the computing system 130 depicted in FIG. 2 includes an arrangement of computer hardware and software components. The computing system 130 may include many more (or fewer) elements than those shown in FIG. 2. It is not necessary, however, that all of these generally conventional elements be shown in order to provide an enabling disclosure.

As illustrated, the computing system 130 includes a processor 160 that is linked to a user interface 170. The user interface 170 includes a graphical display 172 for displaying the retrieved information to the user. The retrieved information may be presented to the user as an ordered list if information. The processor 160 is also linked to a memory 150 that has an engine 180 which stores the various computing modules, programs and software for running the DD system 100. Each of these components may be linked and communicate with one another by way of a communication bus running between the various components and modules. The processor 160 may thus receive information and instructions from other computing systems or services via a network. The processor 160 may also communicate to and from the memory 150 and further provide output information to the graphical display 172. The user interface 170 may accept input from a device such as a keyboard, mouse, digital pen, microphone, touch screen, gesture recognition system, voice recognition system, gamepad, accelerometer, gyroscope, or other input device in order to properly operate the user interface.

The memory 150 may include a variety of storage medium including RAM, ROM and/or other persistent, auxiliary or non-transitory computer-readable media. The memory 150 may store the operating system that provides computer program instructions for use by the processing unit 160 in the general administration and operation of the computing system 130. The memory 150 may further include computer program instructions, such as modules, and other information for implementing aspects of the present disclosure.

The modules may comprise instructions stored in one or more memories and executed by one or more processors. Each memory can be a RAM memory, a flash memory, a ROM memory, an EPROM memory, an EEPROM memory, a register, a hard disk, a removable disk, a CD-ROM, or any other form of storage medium known in the art. Each of the processors may be a central processing unit (CPU) or other type of hardware processor, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. The processor 160 may be a general purpose processor, microprocessor, controller, microcontroller, or state machine. A processor may also be implemented as a combination of computing devices, for example, a combination of a DSP and a microprocessor, a plurality of microprocessors, one or more microprocessors in conjunction with a DSP core, or any other such configuration. Exemplary memories are coupled to the processors such that the processors can read information from and write information to the memories. In some embodiments, the memories may be integral to the processors. The memories can store an operating system that provides computer program instructions for use by the processors or other elements included in the system in the general administration and operation of the DD system 100.

The computing system 130 that is linked to the database 110 and processor 160 includes the engine 180. The engine 180 can include a source data extraction module 182, a page ranking module 184, and an entity analysis module 186, a sentiment analysis module 188, an asset analysis module 190, and a gene homology module 192. In some embodiments, the engine 120 can contain additional modules. In some embodiments, the engine 180 can comprise more than one module performing similar or identical functions to those of modules 182, 184, 186, 188, 190, and 192. In some embodiments, one or more of the modules can be omitted or combined with another module. The engine 180 can access and process information from the database 110. For example, the engine 180 can retrieve data from the source database 112 and/or the gene database 114. The engine 180 can provide one or more outputs, and receive one or more inputs to and from the processor 160 and user interface 170 as described herein.

The engine 180 may be a conventional software package of instructions and processes. In one embodiment, the engine 180 includes the source data extraction module 182. The source data extraction module 182 can extract source data from sources stored in source database 112. Source data extracted can be, for example, metadata. The metadata may include, for example, a candidate name such as a drug name, a molecular compound or formula, a molecular structure diagram, a mechanism of action, a biomolecule such as a protein or enzyme implicated in the indication, a therapeutic target, an indication for animals and/or humans, a form factor, a mode of administration, pharmacokinetics information, toxicology information, adverse effects, patent information such as patent term, intellectual property ownership, researchers, authors, contact information of owners or licensees, phase of clinical testing or regulatory approval, type or class of drug, genetic data associated with the drug, a summary of drug related data, general concerns, efficacy, supporting publications, business funding, business expenditures, design of experiment, results of clinical testing, regulatory submissions, regulatory documentation, and drug vendors. Metadata retrieved by source data extraction module 182 can be made available to other modules such as the page ranking module 184 and the sentiment analysis module 188.

The engine 180 includes a page ranking module 184. The page ranking module 184 can rank sources returned from a user search, for example, sources stored in source database 112. The page ranking module 184 can process information such as metadata retrieved by source data extraction module 182. Pages are ranked by algorithm. The page ranking algorithm may be a weighted combination of algorithms. For example, the page rank may be determined by a combination of an overall page ranking as weighted by an algorithm to determine the similarity of source content to a user's previous searches. The overall page ranking may be determined by, for example, PageRank, originally developed by Google®. The similarity of search content to a user's previous search may be weighted, for example, by Djikstra's algorithm. For example, the average distance from a user's previous searches to the elements in a new search may be calculated, and the page ranked sources in the overall page ranking may be weighted according to the similarity of previous searches. For example, the page ranking module 184 can analyze researchers, such as patent inventors or authors of a scientific publication, for the number of citations on which the researcher is an author. The number of citations may be appended to the source and included in a page ranking algorithm. For example, a source authored by a researcher with a higher number of total citations may be ranked higher in a page ranking.

The engine 180 includes the entity analysis module 186. Entity analysis module 186 can seek connections between entities. For example, following a search by a user, an author of a publication can be analyzed through social networking website information stored in source database 112 to determine if the author is connected to the user. The entity analysis module may then determine a degree of separation from the user and identify a contact through which the user might be connected to the author. The entity analysis module may also retrieve information, such as business entity information, for a relevant entity, such as an owner of intellectual property. In one specific example, a business named as an assignee on a patent may be analyzed for publically available financial information, such as revenue. The entity analysis module may also determine subsidiaries or owners of relevant business entities.

The engine 180 also includes the sentiment analysis module 188. The sentiment analysis module 188 can perform sentiment analysis of a source stored in source database 112. Thus, the sentiment analysis module 188 can analyze a full text source using natural language processing to determine if the authors report a favorable outcome of a clinical or research endeavor. The sentiment may be implemented using a natural language processing algorithm or module as described herein.

The engine 180 includes an asset analysis module 190. The asset analysis module 190 can retrieve and analyze information related to a specific asset such as a drug. The asset analysis module 190 may access information stored in the source database 112, for example, as full text sources. For example, the asset analysis module 190 can retrieve sales information for a drug, for examples revenue from the drug for each year the drug was sold. The asset analysis module 190 can extract regulatory information, such as regulatory approvals. The asset analysis module 190 can extract clinical trial information, such as the number of patients to which the drug has been administered, or the number of adverse events associated with a drug in clinical trials. The asset analysis module 190 can extract patent information, such as remaining patent term, or number of patents that name the asset in a claim. The asset analysis module 190 can extract the identity, number and manufacturers of formulations of a compound. The asset analysis module may receive gene-related information from the gene homology module 192. The gene-related information may include the match level between a gene of interest and a reference gene. The asset analysis module 190 can provide information to the page ranking module 184, which is used therein to determine in part a page ranking of sources.

The engine 180 also includes a gene homology module 192. The gene homology module 192 can retrieve and analyze gene information from the gene database 114. For example, the gene homology module 192 can match a mechanism of action, or a biomolecule such as a protein or enzyme implicated in the indication, to a gene of interest, retrieve the gene of interest and a reference gene from the gene database 114, and compare the gene sequences. From the comparison, the gene homology module 192 can generate gene match information. The gene homology module 192 can additionally analyze coding information included in gene database 114 to discover assets relevant to an indication. For example, gene homology module 192 can compare a protein extracted by source data extraction module 182 with a coding sequence for the protein stored in gene database 114. The gene homology module 192 can determine gene match information for the encoded protein. The gene homology module 192 can provide gene match information to the page ranking module 184 and the asset analysis module 190. The gene match information may be, for example, a gene sequence homology percentage.

In some embodiments, the DD system 100 includes the user interface 170 that provides a means for the user to interact with information, such as a listing of page ranked sources, a source, a metadata annotated source, asset information, entity information, or raw data stored in database 110 or index 120. Information may be presented as a graph. The user interface can be any device which enables visual display and interaction by the user including a touchscreen, smartphone, tablet, laptop, computer, or other type of device. The user interface can be connected to a larger network, such as the internet or a cloud, which can provide one or more components of the DD system described herein such as a database or a module. The user interface can include an graphical display 172 that can provide a visual display of data, such as one or more graphs. The graphical display 172 can change in real time, for example, in response to user input. The inputs can be entered by the user, such as by typing, touching, or clicking to the user interface 170.

The engine 180 may provide an annotated source, e.g., a source annotated with metadata for presentation on graphical display 172. The engine 180 may provide a page ranking of sources as provided herein for presentation on graphical display 172. The engine 180 may provide an asset analysis as provided herein for presentation on graphical display 172. The engine 180 may provide a first reporting as provided herein for presentation on graphical display 172.

### Process Overview

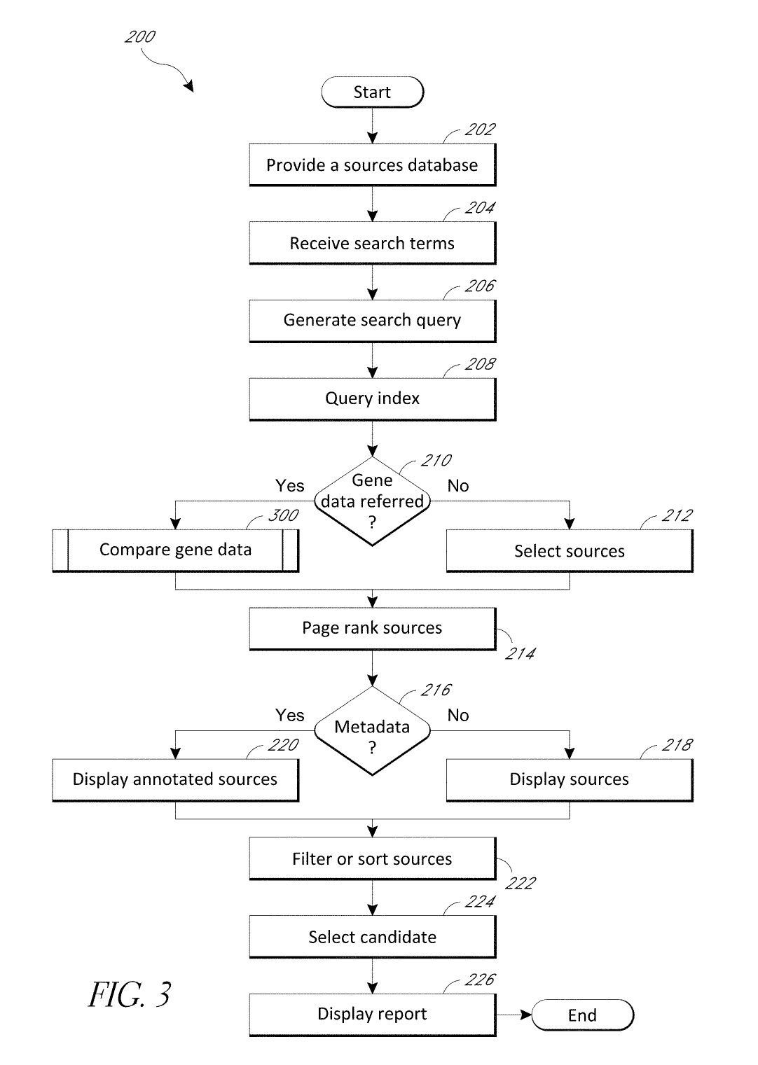

FIG. 3 is a flowchart illustrating an example process 200 carried out, for example, by engine 180 of the DD system 100. The process 200 begins at a start step, and then moves to step 202, wherein a source database 112 is provided. At step 204, the DD system receives search terms, for example, via user interface 170. In some embodiments, the search terms received in step 204 may describe at least an animal of interest. The animal of interest may be identified by, for example, common name, or by taxonomical identifier, for example, species. In some embodiments, the search terms received in step 204 may describe at least an indication and an animal of interest. The indication may be presented by the user as a disease state, symptom, mechanism of action, or other parameter indicating a pathology in the animal of interest. The indication may be a common name for a disease state or pathology. The search terms may also include a biomolecule such as a protein or enzyme implicated in the indication. In some embodiments, the search terms received in step 204 may include key words that appear in a query.

At step 206, engine 180 generates a search query using the received search terms. The search query may include key words. The key words may be the same as the search terms received in step 204. The key words may be based on dictionary correlations of the search terms to other, related terms. For example, a key word may be extracted from a dictionary source referenced by the source data extraction module 182. The key words may be related to the search terms by mere linguistic variation. The key words may be related to the search terms by scientific relationship or scientific equivalence. In some embodiments, engine 180 will return a species as a key word when a search term indicating an animal of the species is received. For example, the search term “dog” may return the key word “canis lupus.” In a further embodiment, engine 180 may return a set of key words corresponding to an indication. In a specific example, a search for the term “leukemia” might return the key words “cancer!” and “tumor!” and “malignan!”. A key word may be a generalization, or may be more specific, relative to the search term. The key words may be, for example, alternative names for a drug, such as generic or proprietary names.

The instructions performed at step 206 may comprise determining alternative search modes. For example, if a user inputs a drug name at step 204, the search query formed at step 206 may include retrieving a chemical structure, or a fragment of a chemical structure, corresponding to the drug name. Alternatively, if a user inputs an indication having a genetic component, such as a disorder arising due to genetic mutation, the search performed at step 206 may include determining a gene sequence of interest to be searched.

After forming the search query at step 206, the process 200 moves to step 208, wherein the engine 180 queries index 120 based on the key words. Sources stored in source database 112 including one or more key words may be discovered by reference to index 120. At step 208, engine 180 may also discover in index 120 a gene of interest stored in gene database 114 related to one or more key words. For example, if a protein is generated as a key word during step 206, the protein may be linked in index 120 to a gene of interest encoding the protein. For further example, a gene of interest may be discovered by reference to index 120 when a key word corresponds to a genetic disorder arising from a gene mutation.

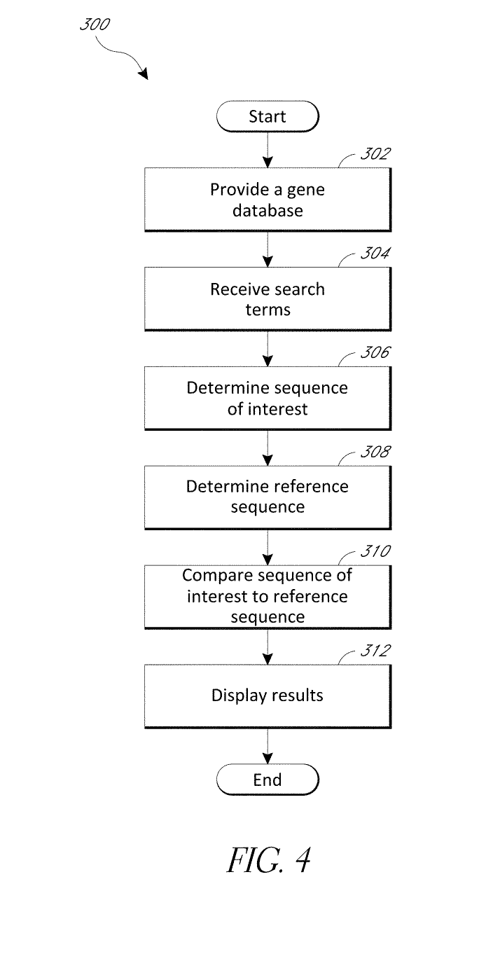

The process 200 then moves to a decision step 210, to determine if gene data was referenced in the query. If a gene of interest was discovered, the process 200 moves to process step 300 wherein the gene homology module 192 will carry out a comparison of a gene of interest to a reference gene. More information can be found on this with reference to FIG. 4. If no gene of interest is discovered at decision step 210, no gene comparison is performed and the process 200 moves to step 212. At step 212, engine 180 selects sources that include one or more key words discovered by reference to index 210 in step 208. Sources selected, for example, full text sources, may be retrieved from source database 112.

The process 200 then moves to a step 214, wherein the engine 180, through the page ranking module 184, page ranks the selected sources. The page ranked sources may be displayed to the user through the graphical display 172. Page ranking may be prioritized according to any factor corresponding to information processed by the engine 180. For example, pages may be ranked base on a patent, regulatory, or social degree of separation factor. The metadata connected with a particular data source or page may be a factor upon which the page rank is sorted. The factors may be weighted and the weighting may be performed according to a trained model. The weighting may be based on user input. For example, a user may request that sources describing assets off patent, or weighted according to least patent term, be prioritized. In such an embodiment, page ranking module 184 may weight the length of a patent more heavily.

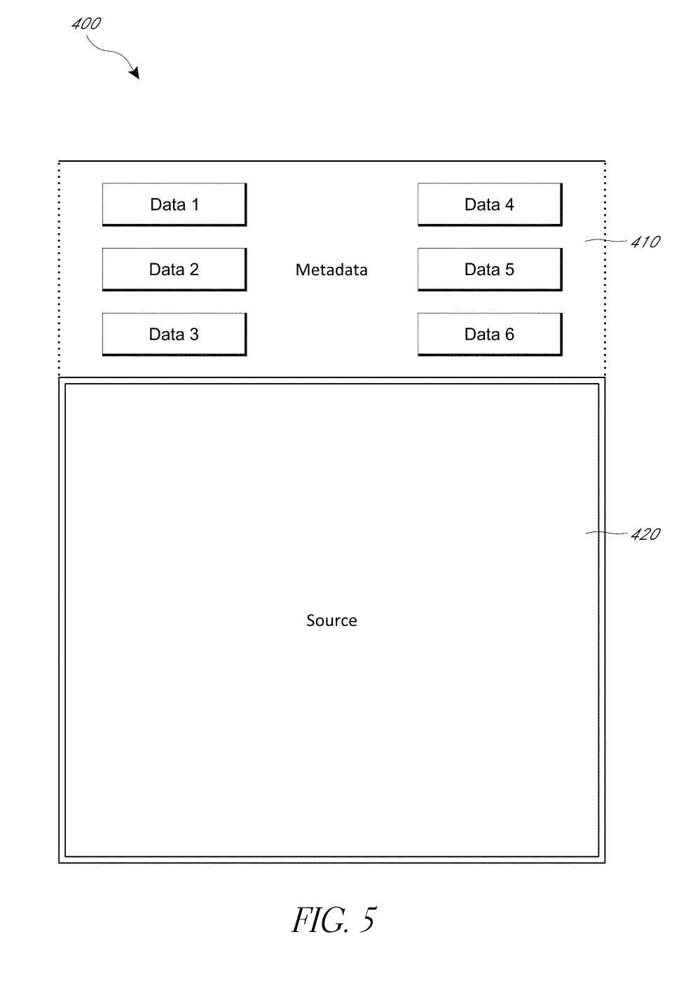

The process 200 then moves to decision step 216 to determine if metadata is available for a source selected or retrieved in step 212. If metadata is available, the process 200 moves to step 220 to display the annotated sources. The annotated source may be as annotated source 400 as depicted in FIG. 5. If a determination is made at the decision step 216 that no metadata is available, the process 200 moves to a step 218 to display the unannotated sources to the user.

After the sources are displayed, the process moves to step 222 wherein the page ranked sources selected at step 212 may be sorted or filtered. For example, sources describing an asset such as a medicine that has not been approved by a regulatory agency may be filtered. Further, the page ranking may be modified in response to a criterion received in a user input. For example, the criterion may be fewest degrees of separation between the user and an author of the source. In such an embodiment, the page ranking module 184 re-ranks the sources according to the criterion. The updated page ranking may be displayed at graphical display 172. The filter parameter may be set by the DD system 100, or be user selectable during the search process.

The process 200 then moves to step 224, wherein a preliminary candidate may be selected by the user. For example, a user may select a candidate, for example an asset, described in a source discovered by engine 180. The candidate may be a medicine in current human use that is desired to be used in veterinary medicine. Engine 180 may include a trained model which selects a candidate automatically.

After a candidate is selected, the process 200 moves to a step 226 wherein a report for the selected candidate may be displayed at graphical display 172. The report may be a first reporting as described herein. The engine 180 collects source data from source database 112. The engine 180 can retrieve source data 112 from the database 110.

In certain implementations, process 200 may further include a step of compiling a custom database. A custom database may be compiled by spider or webcrawler. The custom database may be restricted in subject matter and/or in time. The custom database may target repositories disclosing sources, for example, from a particular field or from a particular institution. For example, the custom database may target journals from a particular field, regulatory information, SEC filings, and/or patent repositories. The repositories may be one or more repositories 10, 12, 14, 16, 18, 20, or 22 described with respect to FIG. 1. In further implementations, process 200 may further include a step of compiling a custom gene database. For example, the custom gene database may include the genome for an animal of interest.

FIG. 4 is a flowchart illustrating an example process 300 carried out, for example, by engine 180 of the DD system 100. At step 302, a gene database, such as gene database 114, is provided. Once the database is provided, the process 300 moves to a step 304, the DD system receives search terms, for example, via user interface 170. In some embodiments, the search terms may include key words that appear in a query, for example, as discussed with respect to step 206 of method 200.

The process 200 then moves to step 306 wherein the engine 180 discovers a relevant gene sequence of interest. The engine 180 may make reference to gene database 114. For example, at step 306, source metadata may be searched for key words related to a gene sequence. For example the key word “hip dysplasia” may correspond to a mutation on a particular animal gene stored in gene database 114. Thus, the animal gene upon which the mutation occurs would be discovered as a gene of interest. At step 308, a reference human gene sequence is identified. Generally, gene database 114 will include information linking the genes of an animal of interest with the genes of a human being. At step 310, the animal gene and the human gene are compared, for example, in the gene homology module 192. A result, for example, as a percentage of gene homology between the animal gene of interest and the reference human gene, is determined. In step 312, the result may be displayed at graphical display 172.

### Annotation System

FIG. 5 is a depiction of an annotated source 400. Annotated source 400 may display metadata 410 and the source 420. For example, source 420 may be a full text source. Source 420 may be a scientific publication, a patent publication, a regulatory submission or report, or a clinical trial report. Metadata 410 may include any metadata described herein, including a candidate name, such as a drug name, a molecular compound or formula, a molecular structure diagram, a mechanism of action, a biomolecule such as a protein or enzyme implicated in an indication, a therapeutic target, an indication for animals and/or humans, a form factor, a mode of administration, pharmacokinetics, toxicology, adverse effects, patent information, intellectual property ownership data, researchers, authors, contact information of owners or licensees, phase of clinical testing or regulatory approval, type or class of drug, genetic data associated with the drug, a summary of drug related data, general concerns, efficacy, supporting publications, business funding, business expenditures, design of experiment, results of clinical testing, regulatory submissions, regulatory documentation, and drug vendors. Patent information metadata may include a patent term for a human medicine to be adapted for animal use.

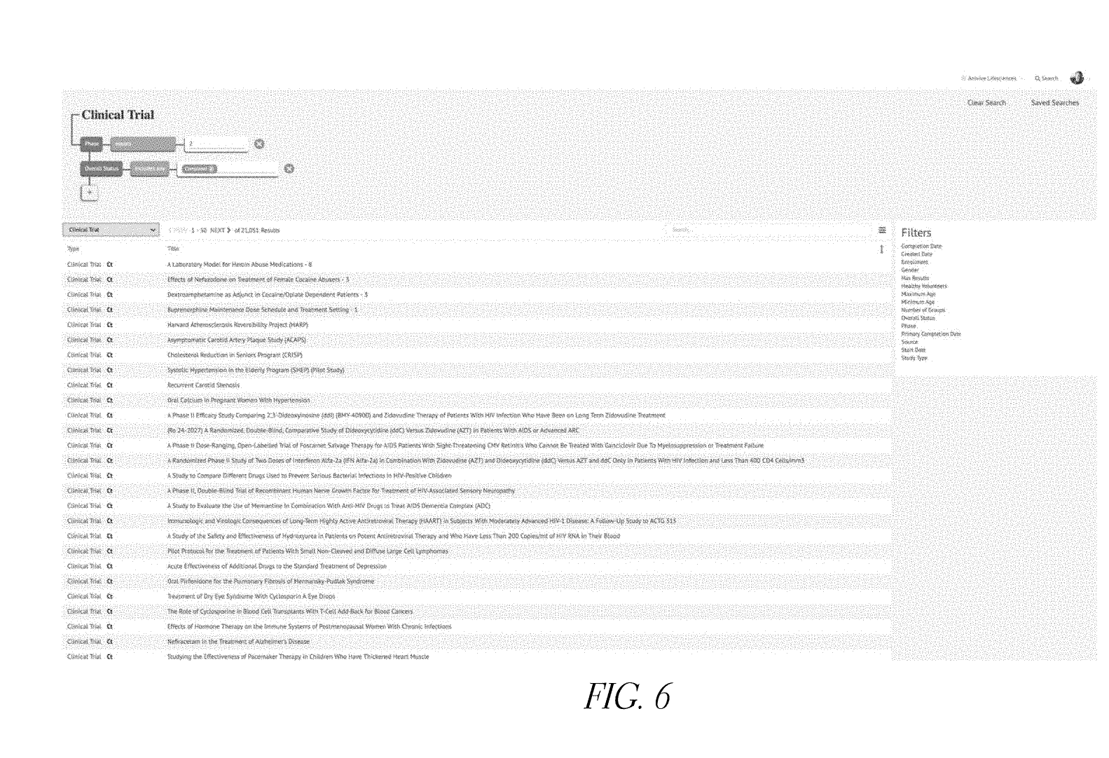

FIG. 6 is an example of a reporting page. In the embodiment of FIG. 6, sources reporting clinical trial data are presented. In FIG. 6, results are filtered to include only sources reporting phase 2 clinical trials, and are further filtered to include only completed trials. A data element displays user selections for filtering sources.

The DD system can utilize many types of data, including, but not limited to patents and patent terms, regulatory status, therapeutic targets, clinical efficacy, safety/toxicology, chemistry, manufacturing and control (CMC), pharmacokinetics, public sentiments, and entities including researchers, owners, assignees, licensees, and the interconnection of such entities via social networks. The DD system database can store sources that include one or more types of data. Generally, each database is indexed. Key words for each source may be stored in the index. In some embodiments, not all types of data will be available for a given source. For instance, as one non-limiting example, ownership data may not be available for a source. Generally, the databases will store sources of information relating to human medicines and the use and study thereof. The databases may also store information relating to animal medicines and the use and study thereof. In some embodiments, the DD allows a user to access a compilation of information relating to the use of human medicines, and evaluate the human medicine for potential veterinary use in a particular animal.

The DD system may also include a gene database. The gene database may store the sequence of bases for a strand of DNA. The gene database may further store information related to downstream associations of the base sequences. For example, the gene database may store information related to base sequences that code a protein. As an additional example, the gene database may store information related to mutations that cause, in whole or in part, a disorder or set of disorders. The disorder may be associated with a medical indication or contraindication. For ease of description, this disclosure describes the DD system with reference to data or information. Reference to “data” or “information” is intended to encompass all types of data.

The DD system can be used by many types of users. The user can be any person or persons, and may be any entity or entities. The DD system can be utilized by a user to understand the information associated with an asset such as a medicine. In particular, the DD system can be used to discover information associated with a human medicine to be adapted for animal use.

As described herein, the DD system can allow the user to visualize metadata associated with a source, and in some embodiments, the source and metadata together, which may be an annotated source. For instance, the DD system can provide a display, juxtaposed with a display of the original source, that illustrates, for example, ownership, potential sales value, patent term, and regulatory information for a source. In some embodiments, the DD system allows a user to visualize data associated with an asset such as a medicine. For instance, the DD system can provide a display that illustrates ownership, potential sales value, patent term, and regulatory information for an asset. The interactive graphical display may provide an intuitive, easy to understand format for such data display.

The DD system can give a user the ability to gain a greater understanding of an individual source. In some embodiments, selecting a source, such as by hovering over or clicking the source, can provide additional information related to the source. The additional information of the source can be viewed on the interactive graphical display of the user interface. The additional information related to the source can allow the user to understand the source. The DD system can give a user the ability to gain a greater understanding of a group of interrelated sources. The DD system can give a user the ability to gain an understanding of a family of related sources, such as one or more families of publications directed to a particular drug, or sharing an author. In some embodiments, the DD system can provide an overview report for families of sources.

The DD system can allow manipulation of the sources and their order of presentation, e.g., their page ranking. The DD system can also allow a user to remove one or more sources from a list returned following a search query. In some embodiments, the user can change a page ranking by inputting a criterion for ranking. As one example, a criterion can be least patent term. As another example, a criterion can be most extensive regulatory approval, for example, approval for human use in the greatest number of jurisdictions. As yet another example, a criterion can be greatest units of sale of an asset such as a medicine. In some embodiments, one or more sources or assets can be removed from the search results by user input selecting sources or assets for removal. For example, patented assets might be removed from a list of sources or assets. In some embodiments, one or more sources or assets can be removed by applying an auto-removal function.

The DD system can provide a verbal, numerical and/or graphical illustration of data. In some embodiments, the DD system can generate scatter plots. For example, the DD system can generate a graph or table.

The DD system can be designed to output an asset recommendation. The asset recommendation can be based on the asset data derived from various sources, compiled, and analyzed by a trained model. In some embodiments, the asset recommendation can be based on one or more types of data provided herein, as extracted from one or more sources.

The DD system can allow the user to gain a better understanding of an asset. The DD system can present metadata for an annotated source. For example, the metadata may reveal a key researcher, a failed business entity, or an asset for which patent term is expired. In some embodiments, the user can select a metadata to be provided with additional information regarding the metadata. The DD system can provide an overview report related to metadata, such as a report of the publications attributed to a particular researcher or assignee.

### Metadata and First Reporting Step

In various embodiments, the first reporting step will display sources, such as patents, and related data such as metadata. The metadata may be related to, for example, the medicines found in the source or the entities associated with the source. The metadata extracted from a source and/or included in a first reporting step may include any type of information provided herein.

The metadata may be patent related data. Patent related data may include any pending US and international patent applications related to each drug, any issued patents related to each drug, the years remaining on each patent, whether the drug is generic, off patent, or public domain, whether there are generic formulations of each drug, when the patents on each drug will expire, and where in the world patents related to each drug have been issued or are pending.

In various embodiments, the metadata visually display geographic data related the drugs located in the search. The geographic data displayed includes the geographic locations of the owners of intellectual property related to the drug, the locations of license holders of the drug, the location where the drug is manufactured, the locations where the drug is undergoing regulatory approval, locations where the drug has received regulatory approval, and locations where the drug is undergoing or has undergone clinical testing.

In various embodiments, the metadata may include data related to the ownership of the drug. For example, the data displayed may include whether the drug is owned by a corporation, a university, or a foundation.

In various embodiments, the metadata may include information about each drug's phase of clinical testing such as whether the drug is in pre-clinical testing, phase I of clinical testing, whether it is approved for a specific use, or whether a clinical study has been completed.

In various embodiments, the metadata may include the drug type of each drug in the search results. For example, the metadata may indicate whether the drug is a small molecule, large molecule or biologic, nutraceutical, or a probiotic or prebiotic.

In various embodiments, the metadata may include the animal related data for each candidate drug in the search results. For example, the report may show what percentage of clinical data is derived from experiments in dogs, cats, rodents, or other species.

In various embodiments, the metadata may include animal safety data, toxicology data, dosage data, pharmacokinetics, drug interactions, adverse effects, and related information.

In some embodiments, the metadata may include efficacy data such as what percentage of the drugs identified during the search have efficacy data associated with them and for which animals efficacy data is available.

In some embodiments, the metadata may include the drug's form factor, for example, whether the drug is available as a tablet, capsule, injectable, eye drop, cream, ointment, or liquid. The results can also display whether the drug is available in regular, quick, or sustained release formulations.

In some embodiments, the metadata may include a value, such as a percentage or a degree of separation, which represents the relationships, if any, between the group or organization to which the user belongs and the people or employees associated with the owner, licensee, or assignee of the drugs identified in the search results. This feature allows the user to determine the relationships that exist between his organization and the company that owns, manufactures, distributes, or licenses the drugs in the search results.

In some embodiments, the metadata may include a value, such as an integer value, a percentage, or a graphical representation of the novelty of the drugs identified during the initial search. Novelty values can be assigned and displayed for each drug individually or to the search results as a whole.

In some embodiments, the metadata may include whether each drug is designated as an orphan drug or regular drug and whether each drug is a minor use drug, whether each drug is intended to be used in minor species, and whether the drug has been registered with the Food and Drug Administration's Center for Veterinary Medicine (CVM). A minor use drug is intended to be used in major species (such as horses, dogs, cattle, pigs, turkeys, and chickens) for diseases that occur infrequently or in limited geographic areas and in small numbers of animals each year. Minor species are all animals other than humans that are not included in the major species. Examples of minor species include ferrets, guinea pigs, zoo animals, parrots, and fish. Some agricultural animals, such as sheep, goats, and honey bees, are considered minor species.

In some embodiments, the metadata may include business entity financial information. For example, metadata extracted or presented with a source may include business funding or business expenditures. In particular, business entity financial information may be retrieved from the Securities and Exchange Comission (SEC) and analyzed or appended to source as metadata.

In some embodiments, the metadata may include information on the conduct of clinical trials. For example, metadata extracted or presented with a source may include design of experiment, or results of clinical testing. In certain embodiments, the metadata may include such information as the number of subjects tested, the length of a study, the geographic locale of the trial, number of adverse events, number of subjects completing the trial, or subject mortality.

In some embodiments, the metadata may include regulatory submissions, or regulatory documentation. For example, metadata extracted or presented with a source may include pharmacology, pharmacokinetics, genotoxicity, reproductive and developmental toxicity, local tolerance, in vitro-in vivo correlation study reports and related information, reports of studies pertinent to pharmacokinetics using human biomaterials, population PK study reports, and related information.

In some embodiments, the metadata may include putative vendors, for example, for a drug candidate. For example, putative chemical suppliers may be discovered via the Chemical Abstracts CHEMCATS® program.

In some embodiments, after the initial results have been displayed in the dashboard or GUI in the first reporting step, and the user has interacted with these data to filter the search results, then intermediate results are generated that show a new set of search results based on user input. In some embodiments the software platform will sort and rank the top 5 or 10 drug candidates for repurposing based on user input provided during the first reporting step. The platform can also rank the top 5 or 10 drugs as if no user input had been provided after the first reporting step. Each drug candidate may be “clickable” wherein clicking on the name of the drug will take the user to a candidate summary page for that drug candidate.

Those having skill in the art will further appreciate that the various illustrative logical blocks, modules, circuits, and process steps described in connection with the implementations disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention. One skilled in the art will recognize that a portion, or a part, may comprise something less than, or equal to, a whole. For example, a portion of a collection of pixels may refer to a sub-collection of those pixels.

The various illustrative logical blocks, modules, and circuits described in connection with the implementations disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or process described in connection with the implementations disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of non-transitory storage medium known in the art. An exemplary computer-readable storage medium is coupled to the processor such the processor can read information from, and write information to, the computer-readable storage medium. In the alternative, the storage medium may be integral to the processor. The processor and the storage medium may reside in an ASIC. The ASIC may reside in a user terminal, camera, or other device. In the alternative, the processor and the storage medium may reside as discrete components in a user terminal, camera, or other device.

Headings are included herein for reference and to aid in locating various sections. These headings are not intended to limit the scope of the concepts described with respect thereto. Such concepts may have applicability throughout the entire specification.

The previous description of the disclosed implementations is provided to enable any person skilled in the art to make or use the present invention. Various modifications to these implementations will be readily apparent to those skilled in the art, and the generic principles defined herein may be applied to other implementations without departing from the spirit or scope of the invention. Thus, the present invention is not intended to be limited to the implementations shown herein but is to be accorded the widest scope consistent with the principles and novel features disclosed herein.

What is claimed is:

1. An electronic system for discovering and evaluating potential veterinary medicines, comprising:
a first database of indexed human medical information;
a processor configured to execute instructions that perform a method comprising:
receiving search terms from a user comprising drug or medical indication data;
generating a first search query from the search terms;
querying the first database to identify candidate human drug information based on the first search query;
analyzing the candidate human drug information to identify animal data relating to the human drug information; and
displaying at least one source of the identified animal data to the user.

2. The system of claim 1, wherein querying the first database to identify candidate human drug information comprises querying a database of human gene information and animal gene information.

3. The system of claim 2, wherein reviewing the candidate human drug information to identify animal data relating to the human drug information comprises comparing the human gene information to the animal gene information.

4. The system of claim 3, wherein the processor is further configured to compare the gene sequence of interest to a reference human gene sequence.

5. The system of claim 1, wherein the processor is further configured to retrieve metadata for the at least one source, and wherein displaying the at least one source comprises displaying a source annotated with metadata.

6. The system of claim 5, wherein the metadata includes one or more information selected from the group consisting of a candidate name, a drug name, a molecular formula, a molecular structure diagram, a mechanism of action, a biomolecule implicated in the medical indication, a therapeutic target, a medical indication for the animal, a medical indication for a human, a form factor, a mode of administration, pharmacokinetics, toxicology, adverse effects, patent information, intellectual property ownership data, researchers, authors, contact information of owners or licensees, a clinical testing report, a phase of regulatory approval, a type or class of drug, genetic data associated with the drug, a summary of drug related data, a sentiment report, efficacy data, supporting publications, business funding, business expenditures, design of experiment, results of clinical testing, regulatory submissions, regulatory documentation, and drug vendors.

7. The system of claim 1, wherein the processor is further configured to receive a drug candidate selection and display metadata associated with the drug candidate.

8. The system of claim 7, wherein the animal data is dog data or cat data.

9. The system of claim 1, wherein the processor is further configured to generate a first page ranking of sources and display the first page ranking.

10. The system of claim 1, wherein the processor is further configured to prepare a meta analysis from metadata for the first source and metadata for the second source, and display a result of the meta analysis.

11. The system of claim 1, wherein the at least one source is selected from the group consisting of a patent source, a news source, a business information source, a clinical trial source, a regulatory source, a dictionary source, and a research publication source.

12. The system of claim 1, wherein the system further comprises an index storing key words for sources in the first database, and wherein querying the first database comprises locating the at least one key word in the index.

13. A method for discovering and evaluating potential veterinary medicines, comprising:
receiving search terms from a user comprising drug or medical indication data;
generating a first search query from the search terms;
querying a first database to identify candidate human drug information based on the first search query;
analyzing the candidate human drug information to identify animal data relating to the human drug information; and
displaying at least one source of the identified animal data to the user.

14. The method of claim 13, wherein querying the first database to identify candidate human drug information comprises querying a database of human gene information and animal gene information.

15. The method of claim 14, wherein reviewing the candidate human drug information to identify animal data relating to the human drug information comprises comparing the human gene information to the animal gene information to determine gene homology between the animal gene data and the human gene data.

16. The method of claim 13, wherein querying a first database comprises querying an index associated with the first database.

17. The method of claim 13, wherein analyzing the candidate human drug information comprises ranking pages of data from the retrieved animal data relating to the human drug information.

18. The method of claim 17, wherein analyzing the candidate human drug information comprises retrieving metadata relating to the candidate human drug data and then displaying that metadata to the user.

19. The method of claim 18, wherein the metadata is selected from the group consisting of a drug candidate name, a drug name, a molecular formula, a molecular structure diagram, a mechanism of action, a biomolecule implicated in the medical indication, a therapeutic target, a medical indication for the animal, a medical indication for a human, a form factor, a mode of administration, pharmacokinetics, toxicology, adverse effects, patent information, intellectual property ownership data, researchers, authors, contact information of owners or licensees, a clinical testing report, a phase of regulatory approval, a type or class of drug, genetic data associated with the drug, a summary of drug related data, a sentiment report, efficacy data, supporting publications, business funding, business expenditures, design of experiment, results of clinical testing, regulatory submissions, regulatory documentation, and drug vendors.

20. The method of claim 13, wherein displaying at the least one source of the identified animal data comprises displaying an ordered list of the identified animal data.
